(19) United States
(12) Patent Application Publication
(10) Pub. No.: US 2013/0253326 A1
(43) Pub. Date: Sep. 26, 2013

(54) METHOD AND SYSTEM FOR INTERFACING HIGH-DENSITY TRANSDUCER ARRAYS

(71) Applicant: David OAKS, TACOMA, WA (US)

(72) Inventor: David OAKS, TACOMA, WA (US)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/427,633

(22) Filed: Mar. 22, 2012

(51) Int. Cl. A61B 8/00

(52) U.S. Cl. 600/459

(57) ABSTRACT

The embodiments of the probe in the ultrasound imaging diagnostic apparatus include at least one rigid interface device for connecting a high-density acoustic array and a predetermined low-density device while maintaining one-to-one connection. The rigid interface provides a first connection area having a predetermined high density and a second connection area having a predetermined medium density that is lower than the high density. The second connection area offers a connection surface for connecting a predetermined low-density device having a predetermined low density that is lower than the medium density.

### FIELD

Embodiments described herein relate generally to ultrasound diagnostic imaging systems for and method of providing an interface for a high-density transducer array in the ultrasound diagnostic imaging system.

### BACKGROUND

In the field of ultrasound medical examination, the acoustic array is ultimately connected to a processing device so as to generate an image based upon the ultrasound that has been detected at the acoustic array. Many acoustic arrays are two-dimensional (2D) in the modern ultrasound imaging systems. Since a number of transducer elements has substantially increased in the 2D arrays, a connection density per a unit area has also substantially increased in the 2D transducer arrays. The increased density causes some difficulty in connecting the high-density transducer arrays with other devices that have generally lower density connections.

There have been some attempts to improve connections between a high-density acoustic array and a predetermined low-density device in the probe of the ultrasound imaging system. In general, the prior art attempts included direct connections between a high-density acoustic array and a predetermined low-density device in the probe. One prior-art attempt provided a plurality of low-density flexible cables for connecting a high-density 2D transducer array. Although the flexible cables such as ribbon cables or flexible printed circuits (FPC) are convenient and inexpensive in connecting a transducer array, the flexible cables takes undesirable amount of physical space in the probe.

In another prior art attempt, multiple FPCs are layered to reduce the physical space for connecting a high-density 2D transducer array. The multi-layered FPCs are also constructed to have interconnections using through holes and via holes. Despite the improved efficiency in space utilization, the multilayered FPCs have gained structural thickness that has often caused acoustic impedance problems among the backing material, acoustic layers and the ultrasound transducer elements. Furthermore, since the multilayered FPCs are substantially rigid in their structure, they have become less convenient in utilizing in the probe.

Yet another prior-art attempt has utilized a pair of flexible cables in combination with an integrated circuit (IC) placed between a 2D transducer and a backing material. The two flexible cables respectively connect to the electrodes on the front and back output surfaces of the acoustic array. By providing the electrodes on the two surfaces, the connection density is lowered to accommodate a low-density device such as the flexible cable. Although the IC connects the two flexible cables using through silicon vias (TSV), since the TSV process require a certain minimum thickness, the IC causes acoustic impedance problems due to the required thickness.

In view of the above described exemplary prior-art attempts, the ultrasound imaging system still needs an improved interface for connecting a high-density acoustic array.

### BRIEF DESCRIPTION OF THE DRAWINGS

FIG. 1 is a schematic diagram illustrating an embodiment of the ultrasound diagnosis apparatus according to the current invention.

FIG. 2 is a diagram illustrating a cross sectional view of a relevant part of one embodiment of the ultrasound probe according to the current invention.

FIG. 3A is a diagram illustrating a top view of one embodiment of a transducer array assembly including the interface device using a printed buildup substrate construction for redistributing the 2D-array connections according to the current invention.

FIG. 3B is a diagram illustrating a cross sectional view of the embodiment of a transducer array assembly at a line A-A of FIG. 3A, including the interface device using a printed buildup substrate construction for redistributing the 2D-array connections according to the current invention.

FIG. 4A is a diagram illustrating a top view of another embodiment of a transducer array assembly including the interface device using a printed buildup substrate construction for redistributing the 2D-array connections according to the current invention.

FIG. 4B is a diagram illustrating a cross sectional view of the embodiment of a transducer array assembly at a line A-A of FIG. 4A, including the interface device using a printed buildup substrate construction for redistributing the 2D-array connections according to the current invention.

FIG. 5 is a diagram illustrating another embodiment of a transducer array assembly including the interface device using a Redistribution Layer (RDL) for redistributing the 2D-array connections according to the current invention.

FIG. 6 a diagram illustrating a yet another embodiment of a transducer array assembly including the interface device using a pair of the Redistribution Layers (RDLs) for redistributing the 2D-array connections according to the current invention.

FIG. 7A is a diagram illustrating a top view of one embodiment of a transducer array assembly including the interface device using a direct layering construction for redistributing the 2D-array connections according to the current invention.

FIG. 7B is a diagram illustrating a cross sectional view of the embodiment of a transducer array assembly at a line A-A of FIG. 7A, including the interface device using a direct layering construction for redistributing the 2D-array connections according to the current invention.

FIG. 7C is a diagram illustrating a cross sectional view of the embodiment of a transducer array assembly at a line A-A of FIG. 7A, including the two interface devices using a direct layering construction for redistributing the 2D-array connections according to the current invention.

### DETAILED DESCRIPTION

According to one embodiment, an ultrasound diagnosis apparatus includes an image creating unit, a calculating unit, a corrected-image creating unit, a hand-free user interface unit and a display control unit. The image creating unit creates a plurality of ultrasound images in time series based on a reflected wave of ultrasound that is transmitted onto a subject from an ultrasound probe. The calculating unit calculates a motion vector of a local region between a first image and a second image that are two successive ultrasound images in time series among the ultrasound images created by the image creating unit. The corrected-image creating unit creates a corrected image corrected from the second image, based on a component of a scanning line direction of the ultrasound in the motion vector calculated by the calculating unit. The s is synonymous with non-touch input device in the current application and interfaces the operator with the ultrasound diagnosis apparatus without physical touch or mechanical movement of the input device. The display control unit performs control so as to cause a certain display unit to display the corrected image created by the corrected-image creating unit.

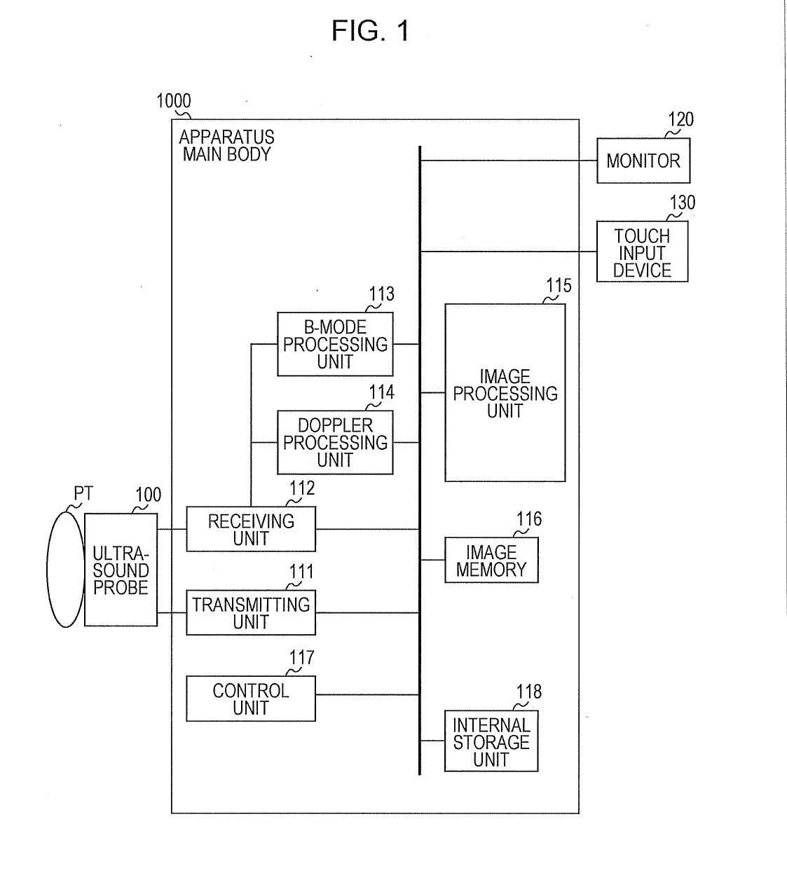

Exemplary embodiments of an ultrasound diagnosis apparatus will be explained below in detail with reference to the accompanying drawings. Now referring to FIG. 1, a schematic diagram illustrates a first embodiment of the ultrasound diagnosis apparatus according to the current invention. The first embodiment includes an ultrasound probe 100, a monitor 120, a touch input device 130, a non-touch input device 200 and an apparatus main body 1000. One embodiment of the ultrasound probe 100 includes a plurality of piezoelectric vibrators, and the piezoelectric vibrators generate ultrasound based on a driving signal supplied from a transmitting unit 111 housed in the apparatus main body 1000. The ultrasound probe 100 also receives a reflected wave from the subject Pt and converts it into an electric signal. Moreover, the ultrasound probe 100 includes a matching layer provided to the piezoelectric vibrators and a backing material that prevents propagation of ultrasound backward from the piezoelectric vibrators.

As ultrasound is transmitted from the ultrasound probe 100 to the subject Pt, the transmitted ultrasound is consecutively reflected by discontinuity planes of acoustic impedance in internal body tissue of the subject Pt and is also received as a reflected wave signal by the piezoelectric vibrators of the ultrasound probe 100. The amplitude of the received reflected wave signal depends on a difference in the acoustic impedance of the discontinuity planes that reflect the ultrasound. For example, when a transmitted ultrasound pulse is reflected by a moving blood flow or a surface of a heart wall, a reflected wave signal is affected by a frequency deviation. That is, due to the Doppler effect, the reflected wave signal is dependent on a velocity component in the ultrasound transmitting direction of a moving object.

The apparatus main body 1000 ultimately generates an ultrasound image. The apparatus main body 1000 controls the transmission of ultrasound from the probe 100 towards a region of interest in a patient as well as the reception of a reflected wave at the ultrasound probe 100. The apparatus main body 1000 includes a transmitting unit 111, a receiving unit 112, a B-mode processing unit 113, a Doppler processing unit 114, an image processing unit 115, an image memory 116, a control unit 117 and an internal storage unit 118, all of which are connected via internal bus.

The transmitting unit 111 includes a trigger generating circuit, a delay circuit, a pulsar circuit and the like and supplies a driving signal to the ultrasound probe 100. The pulsar circuit repeatedly generates a rate pulse for forming transmission ultrasound at a certain rate frequency. The delay circuit controls a delay time in a rate pulse from the pulsar circuit for utilizing each of the piezoelectric vibrators so as to converge ultrasound from the ultrasound probe 100 into a beam and to determine transmission directivity. The trigger generating circuit applies a driving signal (driving pulse) to the ultrasound probe 100 based on the rate pulse.

The receiving unit 112 includes an amplifier circuit, an analog-to-digital (A/D) converter, an adder and the like and creates reflected wave data by performing various processing on a reflected wave signal that has been received at the ultrasound probe 100. The amplifier circuit performs gain correction by amplifying the reflected wave signal. The A/D converter converts the gain-corrected reflected wave signal from the analog format to the digital format and provides a delay time that is required for determining reception directivity. The adder creates reflected wave data by adding the digitally converted reflected wave signals from the A/D converter. Through the addition processing, the adder emphasizes a reflection component from a direction in accordance with the reception directivity of the reflected wave signal. In the above described manner, the transmitting unit 111 and the receiving unit 112 respectively control transmission directivity during ultrasound transmission and reception directivity during ultrasound reception.

The apparatus main body 1000 further includes the B-mode processing unit 113 and the Doppler processing unit 114. The B-mode processing unit 113 receives the reflected wave data from the receiving unit 112, performs logarithmic amplification, envelopes detection processing, and the like so as to creates data (B-mode data) that a signal strength is expressed by the brightness. The Doppler processing unit 114 performs frequency analysis on velocity information from the reflected wave data that has been received from the receiving unit 112. The Doppler processing unit 114 extracts components of a blood flow, tissue, and contrast media echo by Doppler effects. The Doppler processing unit 114 generates Doppler data on moving object information such as an average velocity, a distribution, power and the like with respect to multiple points.

The apparatus main body 1000 further includes additional units that are related to image processing of the ultrasound image data. The image processing unit 115 generates an ultrasound image from the B-mode data from the B-mode processing unit 113 or the Doppler data from the Doppler processing unit 114. Specifically, the image processing unit 115 respectively generates a B-mode image from the B-mode data and a Doppler image from the Doppler data. Moreover, the image processing unit 115 converts or scan-converts a scanning-line signal sequence of an ultrasound scan into a scanning-line signal sequence in a predetermined video format such as television. The image processing unit 115 ultimately generates an ultrasound display image such as a B-mode image or a Doppler image for a display device. The image memory 116 stores ultrasound image data generated by the image processing unit 115.

The control unit 117 controls overall processes in the ultrasound diagnosis apparatus. Specifically, the control unit 117 controls processing performed by the transmitting unit 111, the receiving unit 112, the B-mode processing unit 113, the Doppler processing unit 114, and the image processing unit 115, based on various setting requests input by the operator via the input devices and various control programs and various setting information read from the internal storage unit 118. For Example, the control programs executes certain programmed sequence of instructions for ultrasound transmission and reception, image processing and display processing. The setting information includes diagnosis information such as a patient ID and a doctor's opinion, a diagnosis protocol and other information. Moreover, the internal storage unit 118 is used for storing images stored in the image memory 116 as required. Certain data stored in the internal storage unit 118 is optionally transferred to an external peripheral device via an interface circuit. Lastly, the control unit 117 also controls the monitor 120 for displaying an ultrasound image that has been stored in the image memory 116.

A plurality of input devices exists in the first embodiment of the ultrasound diagnosis apparatus according to the current invention. Although the monitor or display unit 120 displays an ultrasound image as described above, the display unit 120 additionally functions as an input device such as a touch panel alone or in combination with other input devices for a system user interface for the first embodiment of the ultrasound diagnosis apparatus. The display unit 120 provides a Graphical User Interface (GUI) for an operator of the ultrasound diagnosis apparatus to input various setting requests in combination with the input device 130. The input device 130 includes a mouse, a keyboard, a button, a panel switch, a touch command screen, a foot switch, a trackball, and the like. A combination of the display unit 120 and the input device 130 receives predetermined setting requests and operational commands from an operator of the ultrasound diagnosis apparatus. The combination of the display unit 120 and the input device 130 in turn generates a signal or instruction for each of the received setting requests and or commands to be sent to the apparatus main body 1000. For example, a request is made using a mouse and the monitor to set a region of interest during an upcoming scanning session. Another example is that the operator specifies via a processing execution switch a start and an end of image processing to be performed on the image by the image processing unit 115.

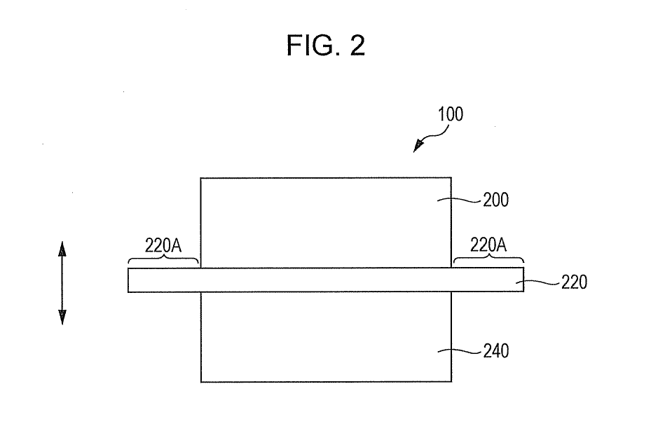

Now referring to FIG. 2, a diagram illustrates a cross sectional view of a relevant part of one embodiment of the ultrasound probe 100 according to the current invention. The exemplary embodiment of the ultrasound probe 100 further includes an acoustic element portion 200, an interface device 220 and a backing portion 240. One implementation of the acoustic element portion 200 is a high-density 2D acoustic array or stack that is comprised of a predetermined number of acoustic elements such as piezoelectric vibrators for generating ultrasound and transmitting towards a patient. The acoustic element portion 200 also receives ultrasound echo or acoustic signals reflected from the patient for converting them to electrical signals. One implementation of the interface device 220 includes a relatively rigid structure that is assembled and later placed immediately adjacent to the acoustic element portion 200. That is, the interface device 200 is embodied in a rigid medium having a rigid interface area for interfacing physical connections. The relatively rigid structure extends beyond the footprint of the acoustic element portion 200 in at least one predetermined lateral direction. One exemplary implementation of the interface device 220 is a printed buildup substrate construction for redistributing the 2D transducer array connections as will be further described. Moreover, the ultrasound probe 100 includes a matching layer provided to the piezoelectric vibrators and a backing material in the backing portion 240 that prevent backward propagation of ultrasound from the piezoelectric vibrators. Although the backing portion 240 is located behind the interface device 220 in the embodiment, the backing portion 240 is not limited to the above relative location with respect to the interface device 220 in other embodiments.

Still referring to FIG. 2, the embodiment of the interface device 220 changes a density of the electrical connections while maintaining the one-to-one connections between each of the electrical output connections from the acoustic element portion 200 and a corresponding one of the electrical output connections such as solder pads located on extended areas 220A. The density of physical electrical connections per a unit surface area is defined to be density according to the current invention. That is, the acoustic array 200 has a predetermined high density for the acoustic elements, and the interface device 220 connects at a high-density level the high-density acoustic array 200 at one end. At the other end, the interface device 220 offers electrical connections at an intermediate density level that is lower than the predetermined high-density while the interface device 220 maintains the individual connections in one-to-one manner from the high-density acoustic array 200. As a result, a low-density device such a flexible ribbon cable or a flexible printed circuit board is advantageously connected to the interface device 220 on the intermediate density side without substantially modifying the electrical connection density level of the low-density device and the high-density device.

Furthermore, because of the simple one-to-one interconnections, the embodiment of the interface device 220 is relatively thin in their structure in a vertical direction of the diagram as indicated by the double-headed arrow. Consequently, the interface device 220 substantially avoids or reduces the acoustic impedance problems of the acoustic array 200 due to its relatively thin structure.

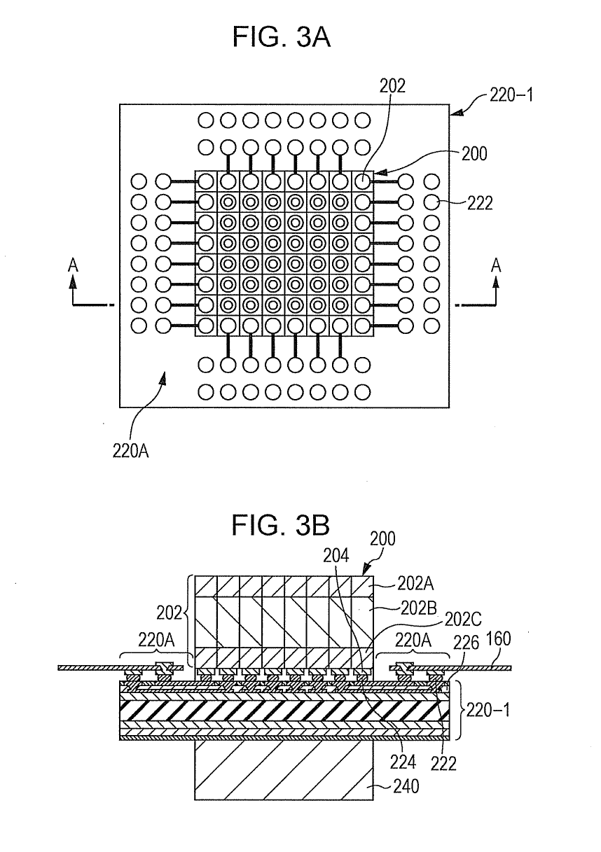

Now referring to FIG. 3A, a diagram illustrates a top view of one embodiment of a transducer array assembly including the interface device using a printed buildup substrate construction for redistributing the 2D-array connections according to the current invention. Although the 2D array 200 has merely eight rows by eight columns of acoustic elements 202 for the purpose of illustration, the 2D array 200 is not limited to any particular size and includes a larger number of the acoustic elements 202 at a high density. The 2D array 200 is fixedly placed on a prefabricated interface device 220-1 using a printed buildup substrate construction such as high-density interconnect (HDI) or high-density packaging (HDP). The prefabricated interface device 220-1 is generally larger than the footprint of the 2D array 200 and extends in four lateral directions in one embodiment. The prefabricated interface device 220-1 is not limited to a particular size or shape in other embodiments.

Still referring to FIG. 3A, the extended portion of the prefabricated interface device 220-1 offers surface areas 220A, where the electrical connections 222 such as solder balls or solder pads are located at a predetermined connection density that is generally lower than the electrical output connection density of the 2D array 200. In the illustrated exemplary embodiment, the prefabricated interface device 220-1 provides the sixty-four electrical connections 222 on a single side of the extended surface areas 220A for the sixty-four acoustic elements 202 of the 8×8 acoustic array 200. The single side is a top surface of the prefabricated interface device 220-1 as will be clearly seen in FIG. 3B. At the same time, the electrical connections are one-to-one between the electrical output connections of the 2D array 200 and the electrical connections 222 of the prefabricated interface device 220-1 as will be also further described.

Now referring to FIG. 3B, a diagram illustrates a cross sectional view of the embodiment of a transducer array assembly at a line A-A of FIG. 3A, including the interface device using a printed buildup substrate construction for redistributing the 2D-array connections according to the current invention. The prefabricated interface device 220-1 is fixedly placed between the 2D array 200 and the backing material 240. The prefabricated interface device 220-1 laterally extends beyond the 2D array 200 and the backing material 240 to provide the extended surface areas 220A in one embodiment. The prefabricated interface device 220-1 is not limited to a particular size, shape or internal structure in other embodiments.

Still referring to FIG. 3B, the prefabricated interface device 220-1 changes the electrical connection density while maintaining the one-to-one connections. The prefabricated interface device 220-1 makes electrical connections at a predetermined high density with the 2D array 200. That is, the high-density connections are made at one end via metal pads 204 of the acoustic elements 202 and inner solder pads 224 of the interface device 220-1. Each of the acoustic elements 202 further includes a matching layer 202A, an ultrasonic transducer 202B such as a PZT or MUT element and a dematching layer 202C. The high-density connections are then redistributed towards the extended surface areas 220A via individual trace 226 in a redistribution layer that is a separately formed substrate-based multilayer. The traces 226 are respectively connected to a corresponding one of outer solder pads 222 at the other end. The outer solder pads 222 are located at a predetermined intermediate density only on a top surface in the extended surface areas 220A. As a result of the redistribution, the high-density connection is converted to the intermediate-density while maintaining the one-to-one connections. A predetermined low-density device 160 such as a flexible cable or a flexible printed circuit is connected to the outer solder pads 222 on the predetermined single surface of the interface device 220-1 in the embodiment according to the current invention. Although the illustrated embodiment provides the intermediate-density outer solder pads 222 on a single surface of the interface device 220-1, other embodiments according to the current invention are not limited to having the outer solder pads 222 on the single surface or all sides.

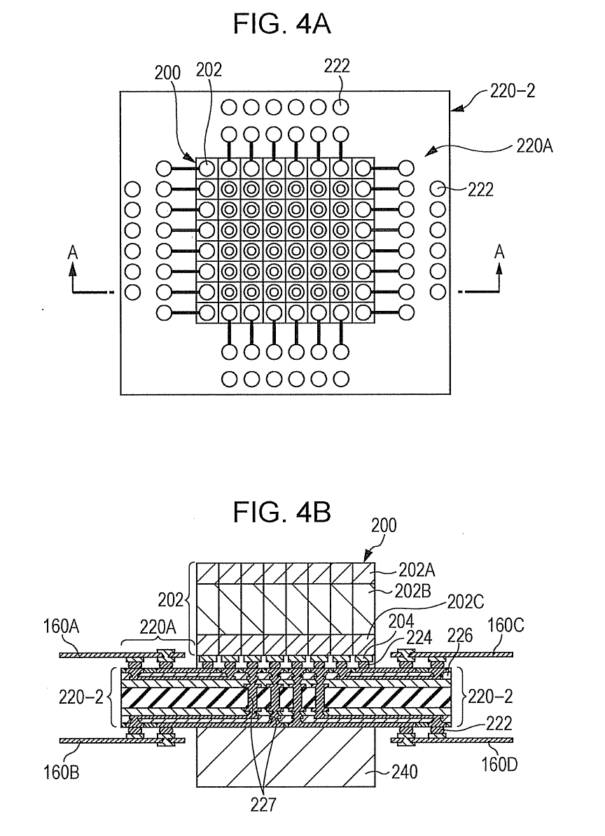

Now referring to FIG. 4A, a diagram illustrates a top view of another embodiment of a transducer array assembly including the interface device using a printed buildup substrate construction for redistributing the 2D-array connections according to the current invention. Although the 2D array 200 has merely eight rows by eight columns of acoustic elements 202 for the purpose of illustration, the 2D array 200 is not limited to any particular size and includes a larger number of the acoustic elements 202 at a high density. The 2D array 200 is fixedly placed on a prefabricated interface device 220-2 using a printed buildup substrate construction such as high-density interconnect (HDI) or high-density packaging (HDP). The prefabricated interface device 220-2 is generally larger than the footprint of the 2D array 200 and extends in four lateral directions in one embodiment. The prefabricated interface device 220-2 is not limited to a particular size or shape in other embodiments.

Still referring to FIG. 4A, the extended portion of the prefabricated interface device 220-2 offers surface areas 220A, where the electrical connections 222 such as solder balls or solder pads are located at a predetermined connection density that is generally lower than the electrical output connection density of the 2D array 200. In the illustrated exemplary embodiment, the prefabricated interface device 220-2 provides fifty-two (14+14+12+12) electrical connections 222 on a top surface of the extended surface areas 220A. Although the top view diagram does not show, the prefabricated interface device 220-2 also provides twelve electrical connections 222 on a bottom surface of the extended surface areas 220A. Thus, the prefabricated interface device 220-2 provides the total of sixty-four connections for the sixty-four acoustic elements 202 for the 8×8 acoustic array 200. At the same time, the electrical connections are one-to-one between the electrical output connections of the 2D array 200 and the electrical connections 222 of the prefabricated interface device 220-2 as will be further described.

Now referring to FIG. 4B, a diagram illustrates a cross sectional view of the embodiment of a transducer array assembly at a line A-A of FIG. 4A, including the interface device using a printed buildup substrate construction for redistributing the 2D-array connections according to the current invention. The 2D array 200 is fixedly placed between the prefabricated interface device 220-2 using a printed buildup substrate construction and the backing material 240. The prefabricated interface device 220-2 laterally extends beyond the 2D array 200 and the backing material 240 to provide the extended surface areas 220A in one embodiment. The prefabricated interface device 220-2 is not limited to a particular size, shape or internal structure in other embodiments.

Still referring to FIG. 4B, the prefabricated interface device 220-2 changes the electrical connection density while maintaining the one-to-one connections. The prefabricated interface device 220-2 makes electrical connections at a predetermined high density with the 2D array 200. That is, the high-density connections are made at one end via metal pads 204 of the acoustic elements 202 and inner solder pads 224 of the interface device 220-2. Each of the acoustic elements 202 further includes a matching layer 202A, an ultrasonic transducer 202B such as a PZT or MUT element and a dematching layer 202C. The high-density connections are then redistributed towards the extended surface areas 220A via an individual trace 226 and or a via 227 in a redistribution layer that is a separately formed substrate-based multilayer. The traces 226 and or the thorough holes 227 are respectively connected to a corresponding one of outer solder pads 222 at the other end. The outer solder pads 222 are located at a predetermined intermediate density on both top and bottom surfaces in the extended surface areas 220A. As a result of the redistribution, the high-density connection is converted to the intermediate-density while maintaining the one-to-one connections. A predetermined low-density device such as a flexible cable 160 is connected to the outer solder pads 222 on the both sides of the interface device 220-2 in the embodiment according to the current invention. The low-density device also includes a printed circuit board that is used in lieu of or in combination with the flexible cable 160. Although the illustrated embodiment provides the intermediate-density outer solder pads 222 on both surfaces of the interface device 220-2, other embodiments according to the current invention are limited to having the outer solder pads 222 on a single surface.

Now referring to FIGS. 3B and 4B, the extended surface areas 220A are optionally implemented in a different size based upon the same predetermined intermediate density according to the current invention. As both sides or surfaces of the extended surface areas 220A are populated with the outer solder pads 222 as in the case of the interface device 220-2, a smaller area is optionally needed in comparison to the single side or surface of the extended surface areas 220A, where the outer solder pads 222 are populated as in the case of the interface device 220-1. Consequently, the overall transducer array assembly size is advantageously reduced in the embodiment of the interface device 220-2. On the other hand, if the interface device 220-2 is optionally implemented using the vias or thorough holes 227, the required thickness of the interface device 220-2 is likely to be larger than the interface device 220-1, which lacks the through holes 227. Furthermore, the first interface device 220-1 and the second interface device 220-2 are generally manufactured using a Flip chip packaging substrate technology. The Flip Chip packaging technology is a low cost high volume process that supports the high density interconnection which is suited for the 2D transducer arrays.

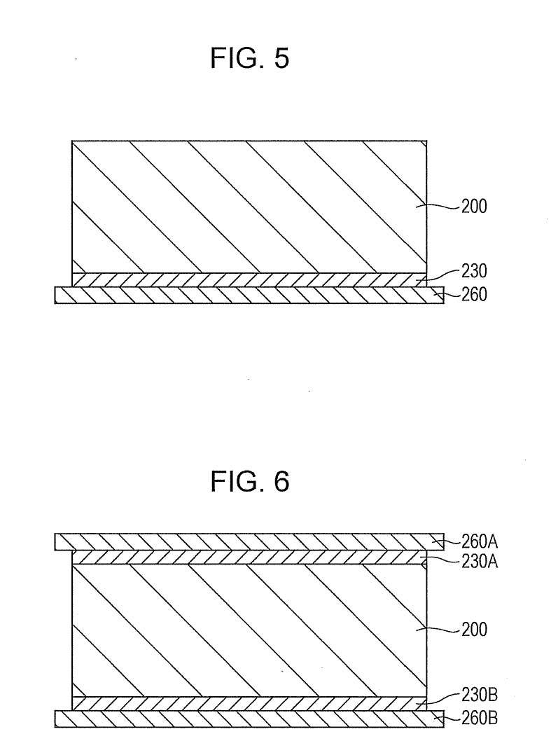

Now referring to FIG. 5, another embodiment of a transducer array assembly including the interface device using a Redistribution Layer (RDL) 230 for redistributing the 2D-array connections according to the current invention. The RDL 230 is used as the interface between the backing or Piezoelectric of the 2D array 200 and a Flip Chip packaging 260. The RDL 230 is another high density interconnection technology that is directly applied to the PZT, Backing or CMUT technology. The redistribution layer is capable of connecting the high density below 1 micron at least on one end. That is, the interface device is embodied in a rigid medium having a rigid interface area for interfacing physical connections. The redistribution layer is capable of connecting the low-density device that is embodied in a flexible medium at the other end.

Now referring to FIG. 6, yet another embodiment of a transducer array assembly including the interface device using a pair of the Redistribution Layers (RDLs) 230A and 230B for redistributing the 2D-array connections according to the current invention. The RDLs 230A and 230B are used as the interface between the backing or Piezoelectric of the 2D array 200 and Flip Chip packagings 260A and 260B. The RDLs 230A and 230E are respectively formed to connect the output terminal or connections on the top and bottom surfaces of the 2D array 200. Furthermore, the RDLs 230A and 230E are directly formed on the top and bottom surfaces before the Flip Chip packaging 260A and 260B are placed on the formed RDLs 230A and 230B. The RDLs 230A and 230B are another high density interconnection technology that is directly applied to the PZT elements, the backing or the CMUT technology. That is, the Redistribution Layers (RDLs) 230A and 23013 are embodied in a rigid medium having a rigid interface area for interfacing physical connections. Although FIG. 6 illustrates the embodiment having a pair of the RDLs 230A and 230B, either one of the RDLs 230A and 230B is formed on the top or bottom surfaces of the 2D array 200 in an alternative embodiment.

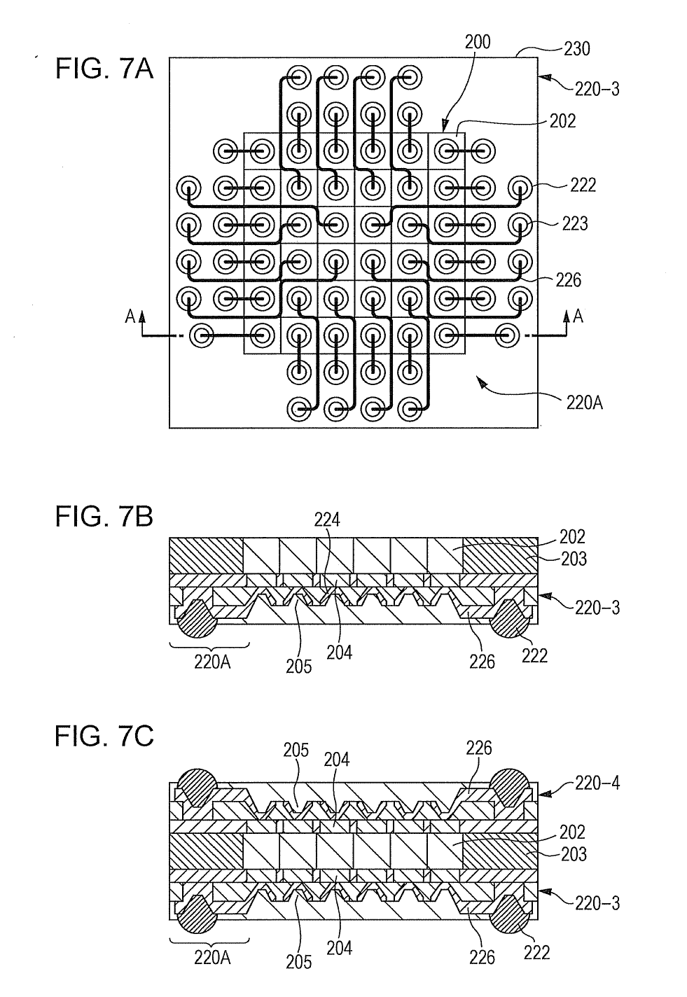

Now referring to FIG. 7A, a diagram illustrates a top view of one embodiment of a transducer array assembly including the interface device using a direct layering construction for redistributing the 2D-array connections according to the current invention. Although the 2D array 200 has merely six rows by six columns of acoustic elements 202 for the purpose of illustration, the 2D array 200 is not limited to any particular size and includes a larger number of the acoustic elements 202 at a high density. Redistribution Layers (RDLs) in an interface device 220-3 are formed directly on the 2D array 200 by layering a set of predetermined materials. That is, the interface device 220-3 is an integrated redistribution layer. Although the fabricated interface device 220-3 is generally larger than the footprint of PZT or MUT elements 202 in the 2D array 200 and extends in four lateral directions in one embodiment, the fabricated interface device 220-3 is not limited to a particular size or shape in other embodiments.

Still referring to FIG. 7A, the extended portion of the fabricated interface device 220-3 offers surface areas 220A, where the electrical connections 222 such as solder balls or solder pads are located at a predetermined connection density that is generally lower than the electrical output connection density of the 2D array 200. In the illustrated exemplary embodiment, the fabricated interface device 220-3 provides the thirty-six electrical connections 222 on the extended surface areas 220A on a single side of the 6×6 acoustic array 200 for the thirty-six acoustic elements 202. The single side is a bottom surface of the fabricated interface device 220-3 as will be clearly seen in FIG. 37B. At the same time, the electrical connections are one-to-one between the electrical output connections of the 2D array 200 and the electrical connections 222 of the fabricated interface device 220-3 as will be also further described.

Now referring to FIG. 7B, a diagram illustrates a cross sectional view of the embodiment of a transducer array assembly at a line A-A of FIG. 7A, including the interface device using a direct layering construction for redistributing the 2D-array connections according to the current invention. The fabricated interface device 220-3 is formed directly on the 2D array 200. Since the fabricated interface device 220-3 is generally larger than the footprint of PZT or MUT elements 202 in the 2D array 200 and extends in four lateral directions in one embodiment, the extended portion of the interface device 220-3 is supported by an extended footprint portion 203 of the 2D array 200. The fabricated interface device 220-3 is not limited to a particular size, shape or internal structure in other embodiments.

Still referring to FIG. 7B, the fabricated interface device 220-3 changes the electrical connection density while maintaining the one-to-one connections. The fabricated interface device 220-3 makes electrical connections at a predetermined high density with the 2D array 200. That is, the high-density connections are made at one end via metal pads 204 of the acoustic elements 202 and inner solder pads 224 of the interface device 220-3. The high-density connections are then redistributed towards the extended surface areas 220A via individual trace 226 in a redistribution layer that is a separately formed substrate-based multilayer. The traces 226 are respectively connected to a corresponding one of outer solder pads 222 at the other end. The outer solder pads 222 are located at a predetermined intermediate density only on the bottom surface in the extended surface areas 220A. As a result of the redistribution, the high-density connection is converted to the intermediate-density while maintaining the one-to-one connections. A predetermined low-density device such as a flexible cable or a flexible printed circuit is connected to the outer solder pads 222 on the predetermined surface of the interface device 220-3 in the embodiment according to the current invention. Although the illustrated embodiment provides the intermediate-density outer solder pads 222 on a single surface of the interface device 220-3, other embodiments according to the current invention are not limited to having the outer solder pads 222 on the single surface or all sides of the interface device 220-3.

Now referring to FIG. 7C, a diagram illustrates a cross sectional view of the embodiment of a transducer array assembly at a line A-A of FIG. 7A, including the two interface devices using a direct layering construction for redistributing the 2D-array connections according to the current invention. A first fabricated interface device 220-3 is formed directly on a bottom surface of the 2D array 200. In addition, a second fabricated interface device 220-4 is formed directly on a top surface of the 2D array 200. That is, the interface devices 220-3 and 220-4 are each an integrated redistribution layer. Since the fabricated interface devices 220-3 and 220-4 are generally larger than the footprint of PZT or MUT elements 202 in the 2D array 200 and extends in four lateral directions in one embodiment, the extended portion of the interface devices 220-3 and 220-4 is supported by an extended footprint portion 203 of the 2D array 200. The fabricated interface devices 220-3 and 220-4 are not limited to be identical with respect to a particular size, shape or internal structure in other embodiments.

Still referring to FIG. 7C, the fabricated interface devices 220-3 and 220-4 change the electrical connection density while maintaining the one-to-one connections. The fabricated interface devices 220-3 and 220-4 make electrical connections at a predetermined high density with the 2D array 200. That is, the high-density connections are made at one end via metal pads 204 of the acoustic elements 202 and inner solder pads 224 of the interface device 220-3. The high-density connections are then redistributed towards the extended surface areas 220A via individual trace 226 in a redistribution layer that is a separately formed substrate-based multilayer. The traces 226 are respectively connected to a corresponding one of outer solder pads 222 at the other end. The outer solder pads 222 are located at a predetermined intermediate density only on the bottom surface in the extended surface areas 220A. As a result of the redistribution, the high-density connection is converted to the intermediate-density while maintaining the one-to-one connections. A predetermined low-density device such as a flexible cable or a flexible printed circuit is connected to the outer solder pads 222 on the predetermined surface of the interface devices 220-3 and 220-4 in the embodiment according to the current invention. Although the illustrated embodiment provides the substantially identical structure between the interface devices 220-3 and 220-4, other embodiments are not limited to have the substantially identical structure between the interface devices 220-3 and 220-4.

Now referring to FIGS. 7B and 7C, the extended surface areas 220A are optionally implemented in a different size based upon the same predetermined intermediate density according to the current invention. As the extended surface areas 220A are populated with the outer solder pads 222 as in the case of the interface devices 220-3 and 220-4 of FIG. 7C, a smaller area is optionally needed in comparison to the extended surface areas 220A of the interface device 220-3 alone of FIG. 7B. Consequently, the overall transducer array assembly size is advantageously reduced in the embodiment of the interface devices 220-3 and 220-4. Furthermore, the first interface device 220-3 and the second interface device 220-4 are generally manufactured using a Flip chip packaging substrate technology. The Flip Chip packaging technology is a low cost high volume process that supports the high density interconnection which is suited for the 2D transducer arrays.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

What is claimed is:

1. A high-density interconnection system, comprising:
a high-density acoustic array device having electrical connections in a unit area at a predetermined high density;
an interface device for connecting said high-density device and having electrical connections at the high density and a predetermined medium density that is lower than the high density while maintaining one-to-one connections of the electrical connections of said high-density acoustic array device, the interface device being embodied in a rigid medium having a rigid interface area for interfacing physical connections; and
a low-density device connected to said interface device at the interface area for having electrical connections at a predetermined low density that is lower than the medium density.

2. The high-density interconnection system according to claim 1 wherein said interface device is located between said high-density acoustic array device and a backing unit.

3. The high-density interconnection system according to claim 1 wherein said rigid interface area extends laterally beyond said high-density acoustic array device.

4. The high-density interconnection system according to claim 1 wherein said interface device includes a redistribution layer that is a separately formed substrate-based multilayer which is connected to said high-density device.

5. The high-density interconnection system according to claim 1 wherein said interface device includes an integrated redistribution layer that is directly formed on said high-density device.

6. The high-density interconnection system according to claim 5 wherein the redistribution layer is capable of connecting the high density below 1 micron.

7. The high-density interconnection system according to claim 1 wherein said low-density device is embodied in a flexible medium.

8. The high-density interconnection system according to claim 7 wherein said low-density device includes a flexible printed circuit.

9. The high-density interconnection system according to claim 1 wherein said interface device has a top surface and a bottom surface and provides the electrical connections at the predetermined medium density on one of the top surface and the bottom surface.

10. The high-density interconnection system according to claim 1 wherein said interface device has a top surface and a bottom surface and provides the electrical connections at the predetermined medium density on both of the top surface and the bottom surface.

11. A method of connecting a high-density device, comprising the steps of:
providing a high-density acoustic array device having electrical connections in a unit area at a predetermined high density;
interfacing the high-density device with an interface device having electrical connections at the high density and a predetermined medium density that is lower than the high density while maintaining one-to-one connections of the electrical connections of the high-density acoustic array device, the interface device being embodied in a rigid medium having a rigid interface area for interfacing physical connections; and
connecting the interface device at the interface area with a low-density device having electrical connections at a predetermined low density that is lower than the medium density.

12. The method of connecting a high-density device according to claim 11 wherein the high-density device includes an acoustic array stack.

13. The method of connecting a high-density device according to claim 11 wherein the interface device includes a redistribution layer that is a separately formed substrate-based multilayer which is connected to said high-density device.

14. The method of connecting a high-density device according to claim 11 wherein the interface device includes an integrated redistribution layer that is directly formed on said high-density device.

15. The method of connecting a high-density device according to claim 14 wherein the redistribution layer is capable of connecting the high density below 1 micron.

16. The method of connecting a high-density device according to claim 11 wherein the low-density device is embodied in a flexible medium.

17. The method of connecting a high-density device according to claim 16 wherein the low-density device includes a flexible printed circuit.

18. A probe, comprising:
a high-density acoustic array having electrical connections in a unit area at a predetermined high density, said high-density acoustic array further including a plurality of acoustic elements, each of the acoustic elements having a matching layer, an ultrasonic transducer and a dematching layer;
an interface device for connecting said high-density device and having electrical connections at the high density and a predetermined medium density that is lower than the high density while maintaining one-to-one connections of the electrical connections of said high-density acoustic array, the interface device being embodied in a rigid medium having a rigid interface area for interfacing physical connections; and
a low-density device connected to said interface device at the interface area for having electrical connections at a predetermined low density that is lower than the medium density.

19. The probe according to claim 18 wherein said rigid interface area extends laterally beyond said high-density acoustic array device.

20. The probe according to claim 18 further comprising a backing unit wherein said interface device is located between said high-density acoustic array device and said backing unit.

21. The probe according to claim 19 wherein said interface device includes a redistribution layer that is a separately formed substrate-based multilayer which is connected to said high-density device.

22. The probe according to claim 19 wherein said interface device includes an integrated redistribution layer that is directly formed on said high-density device.

23. The probe according to claim 22 wherein the redistribution layer is capable of connecting the high density below 1 micron.

24. The probe according to claim 18 wherein said low-density device is embodied in a flexible medium.

25. The probe according to claim 24 wherein said low-density device includes a flexible printed circuit.

26. The probe according to claim 19 wherein said interface device has a top surface and a bottom surface and provides the electrical connections at the predetermined medium density on one of the top surface and the bottom surface.

27. The probe according to claim 19 wherein said interface device has a top surface and a bottom surface and provides the electrical connections at the predetermined medium density on both of the top surface and the bottom surface.
